(19) United States
(12) Patent Application Publication
(10) Pub. No.: US 2010/0215581 A1
(43) Pub. Date: Aug. 26, 2010

(54) CONTRAST AGENTS FOR DETECTING PROSTATE CANCER

(75) Inventor: RALF HOFFMANN, Brueggen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/446,463

(22) PCT Filed: Oct. 15, 2007
(86) PCT No.: PCT/IB07/54183

§ 371 Date: Apr. 21, 2009

(30) Foreign Application Priority Data
Oct. 25, 2006 (EP) 06122907.6

(51) Int. Cl. A61K 49/00; C07K 14/00; C12N 9/96; C07K 16/00

(52) U.S. Cl. 424/9.1; 530/350; 435/188; 530/391.3

(57) ABSTRACT

The invention relates to contrast agents for diagnosing prostate cancer in a human or animal being. These contrast agents are compounds comprising a targeting module that is capable of interacting with a prostate cancer-specific molecular marker and a detectable unit. The invention also concerns the use of such compounds for diagnosing prostate cancer in a human or animal being.

### FIELD OF THE INVENTION

The invention relates to a compound for diagnosing prostate cancer in a human or animal subject wherein the compound comprises a targeting module, which is capable of interacting with a prostate cancer-specific molecular marker, and a detectable unit.

The invention also concerns diagnostic compositions comprising the aforementioned compounds.

Further the invention concerns the use of such compounds and diagnostic compositions in the manufacture of a medicament for diagnosing prostate cancer in a human or animal subject.

The present invention also concerns a method of diagnosing in vivo prostate cancer in the human or animal subject by using the aforementioned compounds and diagnostic compositions.

### BACKGROUND OF THE INVENTION

Prostate cancer is the most common male malignancy and the second leading cause of male cancer-related death in the United States (Jemal et al. (2003) in Cancer Statistics in CA Cancer J. Clin., 53: 5-26). It accounts for 29% of all male cancers and 11% of male cancer-related death. Small prostate carcinomas were detected in 30% of man ages 30 to 49 years, 40% of males over age 50 and 64% of man ages 60 to 70 years (Sakr et al. (1993) in J. Urol., 150: 379-385).

Age adjusted incidents rates increased steadily over the past several decades with dramatic increases associated with the wide spread use of prostate-specific antigen (PSA) screening in the late 1980's and early 1990's.

In general, prostate carcinomas are well-differentiated tumors and normally only slowly growing. In consequence, a lot of silent and latent cases exist. The topology of the prostate carcinomas can be differentiated into four categories:
The silent/latent prostate carcinoma normally remains undiscovered and is often only detected if an autopsy is performed. Therefore, this type of cancer does not play a role in the usual clinical routine.
The incidental prostate carcinoma (T1-tumor) cannot be manifested through clinical investigations, palpation and ultrasonography. It can only be verified by tissue histology upon resection of the prostate. Therapeutic treatment depends on the age of the patient, the tumor stage and grade.
The occult prostate carcinoma occurs clinically and symptomatically as metastasis. There are no symptoms of the primary tumor and the existence of a prostate carcinoma is only possible through the detection of elevated levels of PSA in the metastatic cells. The identification of the primary malignant site is often difficult in particular when the size of the tumor is very small.
The clinical prostate carcinoma (T2-T4 tumors) can be diagnosed by palpation, ultrasonography and by histology. An increased PSA level can be an important indicator for the existence of a clinically manifested prostate tumor.

In view of the above characteristics and incidents rates of prostate cancer an annual screening is generally recommended for all men over the age of 50. Screening on such a large scale requires non-invasive methods of detection and the use of reliable, clinically validated markers.

Currently, prostate cancer screening is based on a combination of three different diagnostic approaches, namely physical examination, biochemical examination and image analysis.

Physical examination is typically done by digital rectal examination (DRE). Digital rectal examination is mandatory in any patient with symptoms of bladder out-flow obstruction. This type of examination is used to assess the size and consistency of the prostate gland. Other approaches include examination of the abdomen by inspection, percussion and palpation in order to detect any distention, palpable renal masses and enlargement of the bladder or tenderness.

While an experienced physician may well be able to detect a tumor by e.g. digital rectal examination, this type of diagnosis is by no means suitable to exclude the presence of particularly early stage tumors let alone to make a clear and reliable distinction between malignant or benign tumors.

Biochemical analysis to a large extent relies on prostate specific antigen serum monitoring. The normally accepted upper level for serum PSA, which is determined by a monoclonal antibody immunoassay, is 4 ng/ml. However, some men with prostate cancer may have normal levels of PSA and elevated levels of PSA may also be due to other factors, such as age, infarction, etc. Therefore, PSA monitoring itself is not sufficient to either exclude or confirm the presence of a tumor. PSA monitoring also does not allow differentiating between malignancy and benignity of a detected tumor.

Hematological measurement may supplement PSA monitoring and include full blood count, plasma viscosity measurements, determination of the erythrocyte sedimentation rate, etc. Again, none of these biochemical approaches can, with certainty, rule out the absence or presence of a tumor.

Therefore, the two aforementioned diagnostic approaches, namely DRE and PSA monitoring must be supported by transrectal ultrasound (TRUS) guided biopsy analysis.

Most men with an abnormal finding on digital rectal examination and/or an elevated PSA value should have a transrectal ultrasound examination with biopsy of any abnormal area of tissue. To this end, the biopsy is usually targeted to a focal area of abnormal echogenicity or to an area of palpable abnormality. As the area of tumor development may not be easily identifiable in the transrectal ultrasound analysis, one will take 12 or sometimes more biopsies from different areas of the prostate during a normal ultrasound scan. These targeted biopsies are then often combined with systematic biopsies in an individual patient which is followed by subsequent histological analysis.

Even though histological analysis, if performed by an experienced histopathologist, may give a good indication of whether a tumor is malignant or benignant, still the morphological manifestation and the expression of some molecular markers in multiple tissue samples obtained from different areas of cancer-bearing prostate vary dramatically. Thus, each biopsy may miss the presence of prostate cancer, including even the most aggressive foci. A TRUS-guided needle biopsy can miss up to 34% of clinically localized prostate cancers and about 10 to 19% of patients with initially negative needle biopsy were diagnosed with prostate cancer on a second biopsy (Keetch et al. (1994) in J. Urol. 151: 1571-1574).

Taken together, none of the aforementioned standard methods are sufficiently sensitive and specific for reliably diagnosing prostate cancer, which makes early detection of this cancer difficult. Early stage prostate tumors are non-palpable by definition and about one third of all prostate tumors are not accessible to DER location. PSA elevation is not specific for cancer; in addition, men with prostate cancer often have normal PSA levels. TRUS guided biopsies may miss relevant tissue sites and lead to false negative results.

Moreover, even if one has been successful in a TRUS-guided biopsy and has obtained tissue samples from tumor tissue, the histological evaluation as to whether the tissue is malignant or benign may be burdensome and will require an experienced, designated histopathologist. The interpretation of samples is made even more complex by the variable normal and malignant histopathology of prostate tissue. In addition to the predominant malignant lesions, i.e. adenocarcinoma, dermal pre-malignant lesions are either found alone or are associated with existing invasive disease. These lesions are grouped under the term “prostate intra-epithelial hyperplasia (PIN)” can vary from low to high grade PIN.

The prognosis for patients with malignant or pre-malignant histopathology will depend to some extent on the grade, size and stage of any invasive tumor present. Histological grading of prostate cancer assesses the glandular differentiation. The most commonly grading system is the so-called Gleason system. The “Gleason score” takes into account inter alia the lack of uniformity. Based on the Gleason score five grades of tumor development can be differentiated on the basis of which a decision as to therapy will be made.

Currently, the situation of prostate cancer diagnosis is such that the total number of detected carcinomas exceeds the number of clinically manifested cases while at the same time there is a significant degree of false negative results. Moreover decision on the malignancy or benignity of a tumor is not straightforward.

In view of the rather burdensome and complicated interplay of diagnostic approaches that have to be undertaken for diagnosing prostate cancer, there is a continuing need for diagnostic methods which increase the likelihood that biopsies may be taken from tumor-afflicted tissues and which support the histopathological decision on malignant and benign tumor tissue.

### OBJECTS AND SUMMARY OF THE INVENTION

It is an object of the present invention to provide compounds, which can be used for diagnosing prostate cancer. It is a further object of the present invention to provide a method of diagnosis for the detection of prostate cancer.

These and other objects of the present invention as they will become apparent from the ensuing description are solved by the subject matter of the independent claims. The dependent claims relate to preferred embodiments of the invention.

The present invention in one aspect relates to a compound for diagnosing prostate cancer in a human or animal subject wherein the compound comprises at least one targeting module and at least one detectable unit with said targeting module being capable of interacting with a prostate cancer-specific molecular marker. Such compounds can function as contrast agents.

Such prostate cancer-specific markers can be selected from the group comprising Chromogranin A (GRN-A), glutathion-S-transferase π (GSTPI), prostate stem cell antigen (PSCA), prostate-specific membrane antigen (PSMA), DD3^{PCA3} (DD3) and telomere reverse transcriptase (telomerase, TERT). A preferred cancer-specific marker is telomerase.

The targeting module can be any molecule that is capable of specifically interacting with the prostate cancer-specific molecular markers. Typically, the targeting module will be at least one molecule being selected from the group comprising antibodies, polypeptides, peptides, peptidomimetics and small organic molecules. Typically, such targeting modules should be capable of easily entering the prostate, the prostate tissue and the prostate cancer afflicted cells. The use of peptides, peptidomimetics and small organic molecules, which are known to easily penetrate across cellular membranes and tissue borders and which interact with the aforementioned prostate cancer-specific molecular markers is preferred. In the case of TERT the targeting module may be preferably selected from small molecule inhibitors such as 3′-azido-2′,3′-dideoxythymidine, disubstituted anthraquinones, fluorenones, acridines, tetracyclic-based compounds, porphyrin-based G-quadruplex inhibitors, and perylenetetracarboxylic diimide.

These compounds are described in detail in Strahl et al. (Mol. Cell. Biol. (1996), 16: 53-56), Sun et al. (J. Med. Chem. (1997), 40: 2113-2116), Perry et al., (J. Med. Chem. (1998), 14:3253-3260), Perry et al. (J. Med. Chem. (1999), 42: 2679-2684), Harrison et al. (Biororgan. Med. Chem. Lett (1999), 9:2463-2568) Perry et al. (Anticancer Drug Des (1999), 14:373-382), Wheelhouse et al. (J. Am. Chem. Soc (1998), 120:3261-3262), Izbicka et al. (Anticancer Drugs (1999), 59: 539-644) and Fedoroff et al. (Biochemistry (1998), 12367-12374). Further references and examples for small molecule inhibitors of TERT can be found in Gowan et al. (Mol. Pharmacology. (2001), 60(5): 981-988) or Fletcher et al. (Expert opinion on therapeutic agents (2001), 5(3): 363-378).

The detectable unit may be any type of molecule that can be detected by well known detection methods, such as magnetic resonance imaging (MRI), by optical detection approaches, such as e.g. microscopy and preferably fluorescence microscopy, by ultrasound, by x-ray detection, by positron emission tomography (PET), by single photon emission computerized tomography (SPECT), by positron emission tomography-computed tomography (PET-CT), etc. Preferred are currently contrast-enhancing materials that can be detected by PET, such as ¹¹C and ¹⁸F, by SPECT, such as ^{99m}Tc, ^{123/5/131}I, ⁶⁷Ga, by optical detection, such as luminescent materials like nanophosphores or semiconducting nanocrystals, carbocyanine dyes, tetrapyrrole-based dyes, delta-amino levulinic acid, fluorescent lanthanide chelates, fluorescein or fluorescein-related fluorophors or by ultrasound imaging, such as (encapsulated gas) bubbles, shell encapsulated droplets or nanoparticles. The use of fluorescein or fluorescein-related and/or derived fluorophors is particularly preferred.

In a particularly preferred embodiment the compounds rely on targeting modules that recognize TERT and are selected from the group consisting of peptides, peptidomimetics and small molecule inhibitors such as 3′-azido-2′,3′-dideoxythymidine, disubstituted anthraquinones, fluorenones, acridines, tetracyclic-based compounds, porphyrin-based G-quadruplex inhibitors and perylenetetracarboxylic diimide. The detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluoresccin.

The present invention also relates to diagnostic compositions which comprise the aforementioned compounds and optionally pharmaceutically acceptable excipients.

Such compositions preferably rely on compounds with targeting modules that recognize TERT and are selected from the group consisting of peptides, peptidomimetics and small molecule inhibitors such as 3′-azido-2′,3′-dideoxythymidine; disubstituted anthraquinones, fluorenones, acridines, tetracyclic-based compounds, porphyrin-based G-quadruplex inhibitors and perylenetetracarboxylic diimide. The detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

Another embodiment relates to the use of the aforementioned compounds in the manufacture of a pharmaceutical composition for diagnosing prostate cancer in a human or animal being. Preferably these pharmaceutical compositions are used for in vivo diagnosis of prostate cancer in a human or animal being. The compound that comprises a targeting module and a detectable marker may be the same as described above. Thus, the detectable targeting module will be capable of interacting with the prostate cancer-specific molecular markers, such as the aforementioned GRN-A, GSTPI, PSCA, PSMA, DD3 and TERT. The targeting module will again be selected preferably from the group comprising antibodies, polypeptides, peptides, peptidomimetics and small organic molecules and the detection unit may be also selected according to the same criteria as mentioned above. Such compositions more preferably rely on compounds with targeting modules that recognize TERT and are selected from the group consisting of peptides, peptidomimetics and small molecule inhibitors such as 3′-azido-2′,3′-dideoxythymidine, disubstituted anthraquinones, fluorenones, acridines, tetracyclic-based compounds, porphyrin-based G-quadruplex inhibitors and perylenetetracarboxylic diimide. The detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

Another embodiment of the present invention relates to a method of diagnosing in vivo prostate cancer in a human or animal subject comprising the steps of:
a) Administration of a compound comprising a targeting module and a detectable marker to said human or animal subject wherein the targeting module is capable of interacting with a prostate cancer-specific molecular marker;
b) Reaction of the targeting module of said compound with the prostate cancer-specific molecular marker;
c) Detection of the interaction between the prostate cancer-specific molecular marker and said compound by measuring a signal which can be generated from the detectable marker; and
d) Deciding on the presence of prostate cancer on the basis of the signal measured in c).

In step c), the signal may preferably be measured outside the human or animal body.

The compound that comprises a targeting module and a detectable marker may be the same as described above. Thus, the detectable targeting module will be capable of interacting with the prostate cancer-specific molecular markers, such as the aforementioned GRN-A, GSTPI, PSCA, PSMA, DD3 and TERT. The targeting module will again be selected preferably from the group comprising antibodies, polypeptides, peptides, peptidomimetics and small organic molecules and the detection unit may be also selected according to the same criteria as mentioned above.

In a preferred aspect these diagnostic methods rely on compounds with targeting modules that recognize TERT and are selected from the group consisting of peptides, peptidomimetics and small molecule inhibitors such as 3′-azido-2′,3′-dideoxythymidine; disubstituted anthraquinones, fluorenones, acridines, tetracyclic-based compounds, porphyrin-based G-quadruplex inhibitors and perylenetetracarboxylic diimide. The detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

Yet another embodiment of the present invention relates to a method of detecting in vivo a prostate cancer-specific molecular marker in the human or animal subject, comprising the steps of:
a) Administration of a compound comprising a targeting module and a detectable marker to said human or animal subject wherein the targeting module is capable of interacting with a prostate cancer-specific molecular marker;
b) Reaction of the targeting module of said compound with the prostate cancer-specific molecular marker; and
c) Detection of the interaction between the prostate cancer-specific molecular marker and said compound by measuring a signal which can be generated from the detectable marker.

The nature of the compound, the targeting module, the detectable unit and the prostate cancer-specific molecular marker may be the same as described above.

### DETAILED DESCRIPTION OF THE INVENTION

As described above, the diagnosis of prostate cancer currently suffers from various drawbacks. Prostate cancer efficiently can only be diagnosed by a combination of methods including typically DRE, PSA serum level monitoring, TRUS-guided biopsy and histological analysis of the biopsy samples. Nevertheless, the TRUS-guided biopsy may lead to false-negative results in approximately 30% of all cases and it is currently difficult to decide early on whether a developing tumor is benign or malignant.

The inventors of the present invention have found that certain molecular markers, which are over-expressed in prostate cancer, can be used to simplify in vivo diagnosis of prostate cancer.

It is known that specific factors may be over-expressed in certain cancer types. This will lead to an increased protein expression level of the specific factor in the tumor tissue and the increased protein level of such a cancer-specific character can be taken as a reliable indicator of ongoing cancer development.

From the publications by Tricoli et al. (Clinical Cancer Research, (2004), 10: 3943-3953, and deKok et al. (Cancer Research, (2002), 62: 2695-2698, it is known that prostate cancer is characterized by increased expression of various molecular markers. The overexpression of chromogranin A (GRN-A), glutathion-S-transferase (GSTPI), PSCA, PSMA, DD3 and TERT is considered to be indicative of prostate cancer development. Particularly, for TERT it is known that it is mainly over-expressed in malignant prostate carcinomas. However, early diagnosis of prostate cancer in vivo on the basis of increased protein levels of these aforementioned factors has not been considered so far.

It is an important aspect of the invention that one can detect the presence of malignant prostate cancer tissue in a patient with high precision as regards the location as well as the benignancy or malignancy of the tumor if one combines a targeting module, which is capable of interacting with a prostate cancer-specific molecular marker, such as the aforementioned proteins with a molecular moiety that can be detected by image analysis technology.

In essence, such compounds constitute contrast agents, that can be used for in vivo image analysis. Because these contrast agents specifically recognize and localize to the prostate cancer-specific molecular targets, they can be used to visualize the tissue areas within the prostate that are likely to contain tumors. As the molecular targets are predominantly expressed in malignant tissue, it is also possible to differentiate between the types of the tumor early on. By detecting such areas of likely ongoing cancer development, it is moreover possible to focus transrectal ultrasound-guided biopsies to these areas of the prostate and thus reduce the likelihood of false negative results as described above. As the compounds can be administered to a human or animal being, it is thus in essence possible to detect prostate cancer in vivo already at an early stage.

The invention in one aspect therefore relates to contrast agents for detecting prostate cancer in the human or animal being. The compounds, which can be used as such contrast agents, comprise at least one targeting module and at least one detectable unit. The targeting module is characterized by its capability to interact with a prostate cancer-specific molecular marker.

The term “prostate cancer-specific molecular marker” refers to any cellular factor that is known to show an increased level during prostate cancer development. The person skilled in the art will understand that such prostate cancer-specific molecular targets may also be over-expressed in other cancer types. Accordingly, the usability or suitability of such a molecular marker will be based on the observation that a marker is observed in increased amounts in prostate carcinogenic tissue in comparison to normal non-carcinogenic tissue. Such markers can be selected form the group consisting of A2M, Akt-1, AMACR, Annexin 2, Bax, Bcl-2, Cadherin-1, Caspase 8, Catenin, Cav-1, CD34, CD44, Clarl, Cox-2, CTSB, Cyclin D1, DD3, DRG-1, EGFR, EphA2, ERGL, ETK/BMK, EZH2, Fas, GDEP, GRN-A, GRP78, GSTP1, Hepsin, Her-2/Neu, HSP27, HSP70, HSP90, Id-1, IGF-1, IGF-2, IGFBP-2, IGFBP-3, IL-6, IL-8, KAI1, Ki67, KLF6, KLK2, Maspin, MSR1, MXI1, MYC, NF-kappaB, NKX3.1, OPN, p16, p21, p27, p53, PAP, PART-1, PATE, PC-1, PCGEM1, PCTA-1, PDEF, P13K p85, PI3K p110, PIM-1, PMEPA-1, PRAC, Prostase, Prostasin, Prostein, PSA, PSCA, PSDR1, PSGR, PSMA, PSP94, PTEN, RASSF1, RB1, RNAseL, RTVP-1, ST7, STEAP, TERT, TIMP 1, TIMP 2, TMPRSS2, TRPM2, Trp-p8, UROC28, VEGF (Tricoli et al., vides supra, deKok et al., vide supra). They can also be selected from the group consisting of PTRF, EB1, Integrin 5 alpha, P13 Kinase, PAK3, ABP280, MCAM, TROY, Myosin VI, AMACR, HSP60, CDK7, TPD52, BRG1, BUB3, PSA, MSH2, GS28, plCln, Aurora kinase A, RBBP, CK1, ERAB, XIAP (Varambally et al., (2005) Cancer Cell, 8, 393-406).

In a preferred embodiment, such a prostate cancer-specific molecular target will be a protein, such as the aforementioned GRN-A, GSTPI, PSCA, PSMA, DD3 and TERT, which are not only be over-expressed in prostate cancer tissue, but to be present mainly in malignant carcinogenic tissue.

A particularly preferred prostate cancer-specific molecular target for detecting prostate cancer is increased expression of TERT. It is known that a lot of cancers including prostate cancer are to some extent characterized in the increased expression of the enzyme telomere reverse transcriptase (telomerase, TERT), which usually ensures genomic stability and integrity in replicating cells, but which is absent in non-replicating cells.

The term “targeting module” relates to any molecular moiety that is capable of specifically interacting with one of the aforementioned prostate cancer-specific molecular target structures. Thus, such targeting modules may be antibodies, which specifically recognize prostate cancer-specific molecular targets, such as GRN-A, GSTPI, PSCA, PSMA, DD3 and TERT. Such antibodies may be of monoclonal or polyclonal origin. If monoclonal antibodies are used, they may be of mouse or rat origin. Antibodies may also be chimeric, humanized antibodies, human antibodies, Fab fragments single-chain antibodies or diabodies or mouse-human chimeric antibodies.

Similarly as antibodies, polypeptides or peptides may be used as the targeting module as long as the (poly)peptides are capable of interacting with a prostate cancer-specific molecular target, such as the aforementioned proteins. The use of polypeptides and peptides that can interact with TERT is particularly preferred.

For the purposes of the present invention, a polypeptide will typically designate polypeptides and proteins, which comprise more than 20 amino acids being linked by peptide bonds. Peptides will comprise between 2 to 19 amino acids being linked by peptide bonds. Preferably, the peptides may be shorter and a preferred length of the peptide will be in the range of approximately 5 to 17 and 10 to 15 amino acids being linked by peptide bonds. It is understood that the terms polypeptides and peptides are not meant to be limited to naturally occurring amino acids. Furthermore, the use of the terms polypeptides and peptides also envisages the use of amino acids, which have been further derivatized to e.g. confer increased stability or new chemical reactivities. The use of glycosylated peptides and polypeptides is also envisaged. The targeting module may also comprise peptidomimetics.

Similarly, the targeting module may be selected from macromolecules, such as hyaluronic acid, apatite and dermatansulfate.

The person skilled in the art may also consider using nucleic acids as targeting modules as long as these nucleic acids are capable of interacting with the prostate cancer-specific molecular targets. Such nucleic acids may be aptamers, antisense DNA/RNA, peptide nucleic acids (PNA), small interfering RNAs, etc. The person skilled in the art will also envisage the use of nucleic acid-derived targeting modules that use other linkages than phosphate bonds in their backbone. Targeting modules which are based on labeled nucleic acids with the label being an MRI contrast agent, an ¹⁹F-magnetic resonance imaging or ¹⁹F-NMR agent, a radiopharmaceutical agent, an ultrasound agent, an optical imaging agent or an x-ray agent and which target TERT do not form part of the invention as far as the contrast agents are concerned. They, however, form part of the invention as far as the use of such compounds in the manufacture of pharmaceutical preparations for diagnosis of prostate cancer or methods of diagnosing prostate cancer are concerned.

Lipids, like phospholipids, lectins, like e.g. leucoside stimulatory lectin and saccharides may also be used as targeting module.

Of course, one may also use small organic molecules that specifically interact with prostate cancer-specific molecular targets, such as those described above. These small organic molecules may be obtained from commercially available compound libraries.

Thus, any type of molecular entity that is capable of interacting with a prostate cancer-specific molecular target can be used as a targeting module. Preferably, the person skilled in the art will consider such targeting modules, which are known to easily penetrate across a cell membrane or tissue borders and which thus can easily access the prostate and tissue of the prostate. For that reason, the use of rather short polypeptides, and particularly peptides, peptidomimetics and organic small molecules, is particularly preferred.

If TERT is used as the prostate cancer-specific molecular target, one can use e.g. small molecule inhibitors of the enzymatic activity of TERT. Such preferred targeting modules include e.g. 3′-azido-2′,3′-dideoxythymidine, disubstituted anthraquinones, fluorenones, acridines, tetracyclic-based compounds, porphyrin-based G-quadruplex inhibitors, and perylenetetracarboxylic diimide.

It is understood that for the case of other prostate cancer-specific molecular targets, such as the aforementioned GRN-A, GSTPI, PRCA, PCMA and DD3, the use of a targeting module, which is made from a peptide or a small organic molecule, is also preferred given that such targeting modules are more likely to easily penetrate into the tissue of the prostate.

The compounds in accordance with the invention, which can be used as a contrast agent for detecting prostate cancer, do not only comprise the above-described targeting module, but also at least one detection unit. This detectable unit may be any molecular moiety that can be detected by molecular imaging technologies as they are known in the art.

For the purposes of the present invention “molecular imaging” is broadly referred to as the characterization and measurement of biological processes and structures in living animals, in organs, in tissue and on the cellular or molecular level. The molecular imaging modalities that can be particularly used for the purposes of the present invention rely on radionuclides, magnetic resonance and optical imaging.

Nuclide medicine techniques include single photon emission computerized tomography (SPECT), positron emission tomography (PET) and positron emission tomography, computed tomography (PET-CT). Optical imaging relies to a large extent on the use of detection units that emit radiation upon suitable excitation, preferably in the visible or ultraviolet spectrum. For optical imaging, one may use microscopy, such as e.g. laser scanning electron microscopy or fluorescence microscopy. The use of optical coherent tomography can also be used for the visual evaluation of individual cells or molecular events, such as the detection of a prostate cancer-specific molecular target as described herein. Thus, for the purposes of the present invention, the detectable unit may be any molecular moiety, that can be detected by the aforementioned techniques, including MRI, PET, SPECT, PET-CT, ultrasound, X-ray and optical methods, such as e.g. fluorescent microscopy. Some of these techniques will be described in more detail.

### Confocal Laser Scanning Microscopy

One of the preferred embodiments of detecting interaction between the targeting module and the prostate cancer-specific molecular marker relies on the use of confocal laser scanning microscopy, which in a further preferred embodiment may use UV light sources. Confocal microscopy offers several advantages over conventional optical microscopy, one of the most important being the elimination of out-of-focus information that distorts the image, controllable depth of field and sub-micron resolution. A further advantage of confocal microscopy is that fluorescence of various portions of the specimen that are out-of-focus can be filtered out and so do not interfere with the portions or sections that are in-focus thereby yielding an image that is considerably sharper and shows a better resolution than a comparable image obtained by classical light microscopy.

The basic principle of confocal scanning microscopy is the use of a screen with a pinhole at the focal point of the microscope lens system which is “conjugate” to the point at which the objective lens is focussed. Only light coming from the focal point of the objective is focussed at the pinhole and can pass through to the detector, which e.g. may be a charge couple device (CCD). Light coming from an out-of-focus section of the sample will be nearly completely filtered out.

Thus, a confocal microscope has a significantly better resolution than a conventional microscope for the x- and y-direction. Furthermore, it has a smaller depth of field in the z-direction. By scanning the focal point through the sample, it is thus possible to view different planes of a sample and to then rebuild a 3-dimensional image of the sample. Furthermore, confocal microscopy is compatible with different wavelengths of light.

If a confocal laser-scanning microscope is integrated in e.g. an endoscopic device, an actuator may be used to scan the confocal microscope over the tissue of interest. A first coarse scan can then be used to determine the morphology of the tissue and the tissue in which an up-regulation of e.g. TERT is seen can be identified from this screen.

For confocal scanning microscope one may use monochromatic or polychromatic light however, monochromatic UV light with a wavelength between 240 nm and 280 nm may be preferred. As a confocal laser scanning microscope one may use a microscope such as LEICA DMLM and having a Qimaging Retiga 2000R FASTCooled Mono 12-bit camera unit (www.qimaging.com) for measuring the signal intensity of the fluorescence signal. A Leica DM6000 may be particularly preferred.

For the purposes of the present invention where cells are to be viewed in their natural tissue environment, the confocal laser-scanning microscope may be integrated into an endoscope. Systems, which are known for this purpose, differ mostly in the manner in which the image is scanned. Two rather advanced commercial systems are available, e.g. from Optiscan and Mauna Kir Technology. The Mauna Kir instrument is a proximally scanned system where the image is transferred down the endoscope with a coherent fibre optic bundle. A selected point or fibre is imaged into the sampled tissue at the distal end. This confocal endomicroscope may be delivered through the working channel of an endoscope. Since the field of view of the endomicroscope is small, placement of the probe is guided by standard video endoscopy. Hence, the endoscope platform must include both a video imager and the confocal microscope. Of course, the endoscope unit may also comprise or be coupled to computer devices and software packages that allow processing of the obtained images.

Detection units may e.g. be Optronics DEI-700 CE three-chip CCD camera connected via a BQ6000 frame-grabber board to computer. Alternatively one may use a Hitachi HV-C20 three-chip CCD camera. Software packages for image analysis may e.g. be the Bioquant True Color Windows 98 v3.50.6 image analysis software package (R&M Biometrics, Nashville, Tenn.) or Image-Pro Plus 3.0 image analysis software. Another system that may be used is the BioView Duet system (BioView Ltd, Rehovot, Israel which is based on a dual mode, fully automated microscope (Axioplan 2, Carl Zeiss, Jena, Germany), an XY motorized 8-slides stage (Marzhauscr, Wetzler, Germany) a 3CCD progressive scan color camera (DXC9000, Sony, Tokyo, Japan) and a computer for control and analysis of the system and the data.

Optiscan has developed an endomicroscope employing distal scanning. In this system, a single fibre transmits light down the endoscope and the fibre end of the distal head is scanned spatially and imaged with optics into the tissue. Optiscan has co-developed with Pentax an endoscope with a confocal microscope integrated into the instrument, thereby freeing up the working channel. Images of tissues that are obtained using this system are of a resolution that allows identification of the localization of cell nuclei.

Other miniature confocal optical devices are described in WO 2004/113962 A2, which is hereby incorporated by reference. Systems for confocal imaging of living tissue are also described in WO 02/073246 A2 as well as in US 2005/0036667 both being also incorporated by reference.

### Optical Coherence Tomography

Yet another possibility of localising cells expressing a prostate cancer-specific molecular marker in vivo in living tissue of a human or animal subject is the use of optical coherence tomography (OCT).

OCT is an imaging technology that achieves up to a few millimetres penetration depth (typically 1.5 to 2 mm) at ultra high resolution (several microns) generating 3-D tissue images in real time. OCT provides 3-D structural images (tissue layers, density changes) for providing spectroscopic information and to achieve functional and molecular imaging at will. OCT is an interferometry-based technology which is capable of measuring signals as small as −90 dB.

A coupler splits light coming from a light source. While one arm serves as a reference arm of the interpherometer, the other one delivers light to the sample and is therefore called the sample arm. The scanning optics provides lateral-scanning capabilities so that the OCT set up obtains an axial scan (A-scan) for each lateral position. All A-scans combined form a 3-D structural image.

When obtaining each A-scan the reference mirror displacement provides depth information. It is also possible to obtain depth information by scanning wavelengths. Several more advance techniques have been developed to achieve higher depth information in shorter times. Spectroscopic OCT is the most advanced among those providing depth scans data without any moving parts.

Currently the fastest OCT system can produce images on around 30 frames per second with more than 1,000 A-scans per frame. Lateral resolution is only limited by the scanning optics and the light focussing system. Axial resolution is in turn light source dependent. A typical axial resolution currently accessible with commercial super luminescence diodes having a bandwidth around 70 nm at 930 nm is approximately 5 μm. One of the best-demonstrated resolutions of around 1.5 μm was achieved by using a Ti:sapphire fs laser.

For the purposes of the present invention, the above described OCT devices and particularly the spectroscopic OCT devices may be miniaturised so that they can be used i.e. in an endoscope system. For miniaturising OCT technology, one may e.g. refer to the proposals made in the publication in Optics Letters 29, 2261 (2004). Typically a miniaturised OCT device, which is capable of being used in an endoscopic system, will provide:
A light source. It determines axial resolution, which is proportional to the source bandwidth. Commercially available SLDs (super luminescence diodes) obtain around 5 μm axial resolution. Better resolution is available if Ti:Sapphire fs laser or Tungsten lamp (very low power) is used (resolution close to 1 μm). A tunable laser is needed for Fourier domain OCT.
Fiber optics components to provide light delivery, fiber coupler and/or circulator to realize Michelson interferometer.
Detection components. This will be dependent on the OCT type. Photodiodes will be typically used, but in the case of spectral OCT spectrally resolved detection will be needed (spectrum analyzer in combination with a liner CCD array).
In case of polarization or phase OCT the engine and the probe components have to be able to maintain light polarization properties.

### 2-Photon Imaging

Of course, cells and tissue in which a prostate cancer-specific marker is up regulated may also be localised using 2-photon imaging technologies. The two-photon method allows real-time three-dimensional in-vivo imaging of tissue. The basic underlying principle is that in this technique, a fluorophore in the tissue is excited by the absorption of two photons of low energy, resulting in the emission of fluorescence. This opposes to conventional (confocal) microscopy, where a single higher-energy photon brings the fluorophore in excitation.

For the two-photon process to occur, a relatively high photon flux is needed, which is generally obtained in the focal point of a confocal microscope. As a consequence, advantages of confocal microscopy also apply to two-photon imaging, like three-dimensional imaging and a high resolution (˜0.2 micron lateral and ˜0.5 micron axial).

Due to the low energy of the excitation photons, single-photon absorption by the tissue is relatively low, which minimizes the amount of photo-induced damage in the tissue. This is a significant advantage for in-vivo applications. Moreover, the low absorption rate leads to a deeper penetration of the photons into the tissue, resulting in an imaging depth up to 0.5 mm.

In conclusion, two-photon imaging combines the advantages of high-resolution confocal microscopy with a large imaging depth and a small amount of photo-induced damage. It allows real-time in-vivo tissue characterization down to cellular level and has been proved to be suitable for diagnosing diseases.

As has been set out above, the detection unit may be any molecular moiety, that can be detected by the aforementioned techniques, including MRI, PET, SPECT, PET-CT, ultrasound, X-ray and optical methods, such as e.g. fluorescent microscopy. Accordingly, the detection unit can be any of the following molecular moieties.

### Magnetic Resonance Imaging

For MRI, the following detection units can be used e.g. ferro-, antiferro-, ferrimagnetic or superparamagnetic materials like iron (Fe), iron oxide γ-Fe₂O₃ or Fe₃O₄ or ferrit with Spinell structure MFe₂O₄ (M=Mn, Co, Ni, Cu, Zn, Cd) or ferrit with granate structure M₃Fe₅O₁₂ (M=Y, La, Ce, Pr, Nd, Sm, Eu, Gd, Tb, Dy, Ho, Er, Tm, Yb, Lu) or ferrit with Magnetoplumbit structure MFe₁₂O₁₉ (M=Ca, Sr, Ba, Zn) or other hexagonal Ferrit structures like e.g. Ba₂M₂Fe₁₂O₂₂ (M=Mn, Fe, Co, Ni, Zn, Mg). In all cases the core can be doped with additional 0.01 to 5.00 mol-% of Mn, Co, Ni, Cu, Zn or F.

One may also use paramagnetic ion (e.g. lanthanide, manganese, iron, copper) based contrast detection units such as gadolinium chelates like Gd (DTPA), Gd (BMA-DTPA), Gd (DOTA), Gd (DO3A); oligomeric structures; macromolecular structures like albumin Gd (DTPA)₂₀₋₃₅, dextran Gd (DTPA), Gd (DTPA)-24-cascade polymer, polylysine-Gd (DTPA), MPEG polylysine-Gd (DTPA), dendrimeric structures of lanthanide based contrast enhancing units, anganese based contrast enhancing units like Mn (DPDP), Mn(EDTA-MEA), poly-Mn(EED-EEA), and polymeric structures as well as liposomes as carriers of paramagnetic ions such as liposomal Gd(DTPA) or non-proton imaging agents;

### Optical Detection

For optical detection one may use luminescent materials like nanophosphores (e.g. rare earth doped YPO₄ or LaPO₄) or semiconducting nanocrystals (so called quantum dots; e.g. CdS, CdSe, ZnS/CdSe, ZnS/CdS), carbocyanine dyes, tetrapyrrole-based dyes (porphyrins, chlorins, phthalocyanines and related structures), delta-aminolevulinic acid, fluorescent lanthanide chelates, fluorescein or 5-aminofluorescein or fluorescein-isothiocyanate (FITC) or other fluorescein-related fluorophors like Oregon Green, Texas Red, Attodye, Cydye, Alexa647, Cy5, Cy3 or naphthofluorescein.

### Ultrasound

For Ultrasound detection one can use shell (e.g. protein, lipid, surfactant or polymer), encapsulated gas (e.g. air, perfluorpropane, dodecafluorcarbon, sulphur hexafluoride, perfluorcarbon), bubbles (like Optison from Amersham, Levovist from Schering); shell (e.g. protein, lipid, surfactant or polymer) encapsulated droplets or nanoparticles (e.g. platinum, gold, tantalum).

### X-Ray

For X-Ray detection one may use iodinated contrast enhancing units like e.g. ionic and non-ionic derivatives of 2,4,6-tri-iodobenzene; barium sulfate based contrast enhancing units; metal ion chelates like e.g. gadolinium based compounds; boron clusters with high proportion of iodine, polymers like iodinated polysaccharides, polymeric tri-iodobenzenes, particles from iodinated compounds displaying low water solubility, liposomes containing iodinated compounds, iodinated lipids like triglycerides or fatty acids.

### PET

For PET-analysis one may use ¹¹C, ¹³N, ¹⁵O, ^{66/8}Ga, ⁶⁰Cu, ⁵²Fe, ⁵⁵Co, ^{61/2/4}Cu, ^{62/3}Zn, ^{70/1/4}As, ^{75/6}Br, ⁸²Rb, ⁸⁶Y, ⁸⁹Zr, ¹¹⁰In, ^{120/4}I, ¹²²Xe and ¹⁸F based tracers like e.g. ¹⁸F-FDG (glucose metabolism), ¹¹C-Methionine, ¹¹C-Tyrosine, ¹⁸F-FMT, ¹⁸F-FMT or ¹⁸F-FET (amino acids), ¹⁸F-FMISO, ⁶⁴Cu-ATSM (hypoxia), ¹⁸F-FLT, ¹¹C-Thymidine, ¹⁸F-FMAU (proliferation).

### SPECT

For SPECT one may use contrast-enhancing units based on radionuclides like e.g. ^{99m}Tc, ^{123/5/131}I, ⁶⁷Cu, ⁶⁷Ga, ¹¹¹In and ²⁰¹Ti.

Besides the above-mentioned markers, one may use e.g. toxins, radioisotopes and chemotherapeutics, UV-C emitting nanoparticles like e.g. YPO₄:Pr, photodynamic therapy (PDT) agents like e.g. compounds based on expanded porphyrine structures or nucleotides for radiotherapy like e.g. ¹⁵⁷Sm, ¹⁷⁷Lu, ^{212/3}Bi, ^{186/8}Re, ⁶⁷Cu, ⁹⁰Y, ¹³¹I, ^{114m}In, At, Ra, Ho.

Alternatives are also smart contrast enhancing units like e.g. chemical exchange saturation transfer (CEST), thermosensitive MRI contrast agents (e.g. liposomal), pH sensitive MRI contrast agents, oxygen pressure or enzyme responsive MRI contrast agents, metal ion concentration dependent MRI contrast agents. Preferably, the detection units for magnetic resonance imaging purposes will be selected from ferro-, antiferro-, ferri- or superparamagnetic materials like iron (Fe), iron oxide γ-Fe₂O₃ or Fe₃O₄. Paramagnetic ion (e.g. lanthanide, manganese, iron, copper) based contrast detection units such as gadolinium chelates like Gd (DTPA), Gd (BMA-DTPA), Gd (DOTA), Gd (DO3A) are also preferred.

For optical detection approaches, the use of the following detection units is particularly preferred: luminescent materials like fluorescein or 5-aminofluorescein or fluorescein-isothiocyanate (FITC) or other fluorescein-related and/or -derived fluorophors like Oregon Green, Texas Red, Attodye, Cydye, Alexa647, Cy5, Cy3 or naphthofluorescein.

In a particularly preferred embodiment of the invention, the contrast agent will be made from a targeting module that recognizes TERT. Preferably, the person skilled in the art will consider such targeting modules, which are known to easily penetrate across a cell membrane or tissue and which thus can easily access the prostate and tissue of the prostate. For that reason, the use of rather short polypeptides, and particularly peptides and organic small molecules such as 3′-azido-2′,3′-dideoxythymidine, disubstituted anthraquinones, fluorenones, acridines, tetracyclic-based compounds, porphyrin-based G-quadruplex inhibitors and perylenetetracarboxylic diimide is preferred. The detectable unit is preferably selected from the group consisting of ¹¹C, ¹⁸F, ^{99m}Tc, ^{123/5/131}I, ⁶⁷Ga, luminescent materials like nanophosphores, semiconducting nanocrystals, carbocyanine dyes, tetrapyrrole-based dyes, delta-aminolevulinic acid, fluorescent lanthanide chelates, fluorescein or other fluorescein-related and/or derived fluorophores, encapsulated gas or bubbles, shell encapsulated droplets or nanoparticles. The detectable unit is particularly preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

It is understood that for the case of other prostate cancer-specific molecular targets, such as the aforementioned GRN-A, GSTPI, PSCA, PCMA and DD3, the use of a targeting module, which is made from a peptide or a small organic molecule, is also preferred given that such targeting modules are more likely to easily penetrate into tissue of the prostate. Again, the detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

The person skilled in the art is clearly aware that a contrast agent in accordance with the invention may not only comprise one targeting module. It may also comprise two, three, four targeting modules. The targeting modules can specifically bind to the same or different prostate cancer-specific molecular marker(s). If more than one targeting module is used and if all targeting module recognizes the same prostate cancer-specific molecular target, the specificity of the contrasting agent can be increased. This may particularly be the case if the targeting modules recognize different structures within the molecular maker. If targeting modules are used that are specific for different prostate cancer-specific molecular markers, selectivity may be increased if the concomitant presence of the different molecular targets is indicative of e.g. malignant prostate cancer development. The concomitant presence can be verified by obtaining e.g. differently localized signals within the same cell.

Similarly the person skilled in the art is aware that a contrast agent can comprise more than one detectable unit such as e.g. two, three, four or more detectable units. The use of more than one detectable unit can increase sensitivity of the measurement. Of course, one may use different types of detectable units so that results from MRI can be compared and eventually be verified by optical measurements.

The person skilled in the art will also consider a combination of the above-mentioned approaches and thus use e.g. simultaneously one contrast agent which is formed from a targeting module being specific for a prostate cancer-specific molecular marker and a detectable unit, and another contrast agent which is formed from a targeting module recognizing another prostate cancer-specific molecular marker and a detectable unit that relies on a different detection principle. Administration and measurement of such combinations of contrast agent will allow detecting prostate cancer by independent approaches in a single experiment.

The person skilled in the art is aware that the targeting module and the detectable unit may be combined by different ways within a single compound. For example, the targeting module may be a small organic molecule, which is linked to a fluorescent marker via a bond. Similarly, the targeting module may be a polypeptide that is linked to any of the aforementioned MRI agents. The linkage between the targeting module and the detection unit may be covalent, but it may also be an ionic bond. Furthermore, the detection unit may be directly linked to the targeting module or it may be separated from the targeting module by a spacer. The person skilled in the art is familiar with linking the aforementioned detectable units to a targeting module such as a polypeptide. For example, the chemical linkage can be provided by e.g. acyl fluoride functionalities in the detectable unit. Other covalent attachment chemistries are also applicable but not limited to anhydrides, epoxides, aldehydes, hydrazides, acyl azides, aryl azides, diazo compounds, benzophenone, carbodiimide, imidoesters, isothiocyanates, NHS esters, CNBr, maleimides, tosylates, tresyl chloride, maleic acid anhydrides and carbonyldiimidazole. The linkage may also be established by homo and/or hetero-bi and/or multifunctional cross-linking agents. Typical cross-linking agents include but are not limited to bis(sulfosuccinimid) bis(diazo-benzidine), dimethyl adipimidate, dimethyl pimelimidate, dimethyl suberaimidate, disuccininnclyl suberate, glutaraldehyde, N-maleimidobenzoyl-N-hydroxysuccinimide, sulfosuccinidyl 4-(N-maleimidomethyl)cyclohexane-1-carboxylate etc.

The contrast agents or compounds in accordance with the invention may contain one or more of the following functional groups: alcohols, phenols, esters, including ester with other acids, carboxylic acids, amides, amines, mercapto-groups, aromatic rings and heterocyclic ring systems. The overall structure of the compounds can be cyclic or linear.

The compounds may carry an overall charge. In such a case the compounds may be used in the form of the salt of the physiological acceptable counter ion, for example an ammonium ion, substituted ammonium ion, alkali metal or alkaline earth metal cation, or an anion derived from an inorganic or organic acid.

It has been set out above that the present invention also relates to pharmaceutical compositions for diagnostic purposes, namely the detection of prostate cancer. Such pharmaceutical compositions that will be referred to also as diagnostic compositions will comprise the above described compounds and optionally pharmaceutically acceptable excipients.

It is understood that these diagnostic preparations will comprise the aforementioned contrast agents and preferably the preferred forms of these contrast agents. Thus, the diagnostic compositions will preferably comprise a contrast agent which is made from a targeting module that recognizes TERT. The person skilled in the art will again preferably consider such targeting modules which are known to easily penetrate across a cell membrane or tissue and which thus can easily access the prostate and tissue of the prostate. For that reason, the use of rather short polypeptides, and particularly peptides and organic small molecules such as 3′-azido-2′,3′-dideoxythymidine; disubstituted anthraquinones, fluorenones, acridines, tetracyclic-based compounds, porphyrin-based G-quadruplex inhibitors and perylenetetracarboxylic diimide is particularly preferred. In these cases, the detectable unit is preferably selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

The contrast agents can be preferably formulated in conventional pharmaceutically or veterinary parenteral administration forms, e.g. suspensions, dispersions, etc., for example aqueous vehicles, such as water or buffers for injections. The contrast agents may also further contain pharmaceutically acceptable diluents and formulation aids, for example lubricants, plasticizers, antioxidants, osmolality adjusting agents, buffers or pH adjusting agents. One of the most preferred formulations of the contrast agents in accordance with the invention includes a sterile solution or suspension for parenteral administration or for direct injection into an area of interest.

While the contrast agents of the present invention may be formulated such that parenteral administration thereof into the vasculature or directly into an organ or muscle tissue is preferred, intravenous administration may be especially preferred. Administration via a non-parenteral route may also be envisaged and includes e.g. transdermal, nasal, oral, buccal and sub-lingual administration or administration into a body cavity, such as e.g. the gastrointestinal tract, the bladder, the uterus or the vagina. The present invention is deemed to cover such administration.

As has been set out above, the compounds and diagnostic compositions in accordance with the invention can be used to detect carcinogenic tumor formation in the prostate at an early stage.

Thus, the present invention also relates to the use of at least one such compound in the manufacture of a pharmaceutical composition for diagnosing prostate cancer in a human or animal subject. Preferably, such pharmaceutical compositions are used for in vivo diagnosis of prostate cancer in a human or animal body.

The above-described preferred compounds are also preferred for use in the manufacture of such pharmaceutical compositions. Thus, a compound, which comprises a targeting module that is capable of interacting with TERT and which may be made from a peptide or small molecule inhibitor, such as 3′-azido-2′,3′-dideoxythymidine; disubstituted anthraquinones, fluorenones, acridines, tetracyclic-based compounds, porphyrin-based G-quadruplex inhibitors and perylenetetracarboxylic diimide and which further comprises a detection unit selected from the group consisting of fluorescein or other fluorescein-related and/or derived fluorophors such as 5-aminofluorescein or fluorescein-isothiocyanate (FITC), Oregon Green, Texas Red, Attodye, Cydye, Alexa647, Cy5, Cy3 or naphthofluorescein.

Other embodiments of the present invention relate to a method of diagnosing in vivo prostate cancer in a human or animal subject, comprising the steps of:
a) Administration of a compound comprising a targeting module and a detectable unit to said human or animal subject wherein the targeting module is capable of interacting with a prostate cancer-specific molecular marker;
b) Reaction of the targeting module of said compound with the prostate cancer-specific molecular marker;
c) Detection of the interaction between the prostate cancer-specific molecular marker and said compound by measuring a signal generated by the detectable unit upon suitable excitation; and
d) Deciding on the presence of prostate cancer on the basis of the signal measured in c).

In step c) measurements of the signal may preferably be done outside the human or animal body.

The person skilled in the art is aware that one typically will use signals from cells which are clearly non-carcinogenic as standard reference for deciding on whether a detected signal is indeed indicative of ongoing cancer development.

Yet another embodiment of the present invention relates to a method of detecting a prostate cancer-specific molecular target in a human or animal subject, comprising the steps of:
a) Administration of a compound comprising a targeting module and a detectable unit to said human or animal subject wherein the targeting module is capable of interacting with a prostate cancer-specific molecular marker;
b) Reaction of the targeting module of said compound with the prostate cancer-specific molecular marker; and
c) Detection of the interaction between the prostate cancer-specific molecular marker and said compound by measuring a signal generated by the detectable unit upon suitable excitation.

It is understood that the compounds that are used in the aforementioned method of diagnosis and method of detecting a prostate cancer-specific molecular target comprise the same compounds and diagnostic compositions as mentioned above. Similarly, the above-mentioned preferred contrast agents and diagnostic compositions will also preferably be used in the aforementioned method of diagnosis and detection of prostate cancer-specific molecular targets. A particularly preferred compound will be one that has a targeting module being made from a peptide or small molecule inhibitor of TERT and a detection unit being such as those described above.

The present invention also relates to a method of producing the aforementioned contrast agents and diagnostic compositions.

The invention has been described above with respect to some preferred embodiments. This, however, is not meant to limit the invention in any way, and the person skilled in the art will be clearly in a position to identify further embodiments that are within the scope and spirit of the invention.

1. Compound for diagnosing prostate cancer in a human or animal subject, wherein the compound comprises at least one targeting module and at least one detectable unit with the targeting module being capable of interacting with a prostate cancer-specific molecular marker.

2. Compound according to claim 1,
wherein the prostate cancer-specific marker is selected from the group comprising chromogranin A (GRN-A), glutathion-S-transferase π (GSTPI), prostate stem cell antigen (PSCA), prostate-specific membrane antigen (PSMA), DD3^{PXA3} (DD3) and telomerase reverse transcriptase (TERT).

3. Compound according to claim 1,
wherein the targeting module is selected from the group comprising antibodies, polypeptides, peptides, peptidomimetics and small organic molecules.

4. Compound according to claim 3,
wherein the targeting module is a small molecule inhibitor of TERT being preferably selected from the group consisting of 3′-azido-2′,3′-dideoxythymidine, disubstituted anthraquinones, fluorenones, acridines, tetracyclic-based compounds, porphyrin-based G-quadruplex inhibitors and perylenetetracarboxylic diimide.

5. Compound according to claim 1,
wherein the detectable unit is capable of being detected by methods selected from the group comprising magnetic resonance imaging (MRI), optical detection such as fluorescence microscopy, ultrasound, x-ray detection, positron emission tomography (PET), single photon emission computerized tomography (SPECT) and positron emission tomography-computed tomography (PET-CT).

6. Compound according to claim 5,
wherein the detectable unit is selected from the group comprising ¹¹C, ¹⁸F, ^{99m}Tc, ^{123/5/131}I, ⁶⁷Ga, luminescent materials like nanophosphores, semiconducting nanocrystals, carbocyanine dyes, tetrapyrrole-based dyes, delta-aminolevulinic acid, fluorescent lanthanide chelates, fluorescein or other fluorescein-related and/or derived fluorophores, encapsulated gas or bubbles, shell encapsulated droplets or nanoparticles.

7. Compound according to any of claims 1 to 6,
wherein the prostate cancer-specific marker is selected from the group consisting of chromogranin A (GRN-A), glutathion-S-transferase π (GSTPI) prostate stem cell antigen (PSCA), prostate-specific membrane antigen (PSMA), DD3^{PCA3} (DD3) and telomerase reverse transcriptase (TERT) and wherein the detectable unit is selected from the group consisting of fluorescein or other fluorescein-related and/or -derived fluorophors such as 5-aminofluorescein or fluorescein-isothiocyanate (FITC), Oregon Green, Texas Red, Attodye, Cydye, Alexa647, Cy5, Cy3 or naphthofluorescein.

8. Compound according to any of claims 1 to 6, wherein the prostate specific marker is TERT and wherein the detectable unit is selected from the group consisting of ¹¹C, ¹⁸F, ^{99m}Tc, ^{123/5/131}I, ⁶⁷Ga, luminescent materials like nanophosphores, semiconducting nanocrystals, carbocyanine dyes, tetrapyrrole-based dyes, delta-amino levulinic acid, fluorescent lanthanide chelates, fluorescein or other fluorescein-related and/or -derived fluorophores, encapsulated gas or bubbles, shell encapsulated droplets or nanoparticles.

9. A compound according to claim 8,
wherein the targeting module is a small molecule inhibitor of TERT being preferably selected from the group consisting of 3′-azido-2′,3′-dideoxythymidine, disubstituted anthraquinones, fluorenones, acridines, tetracyclic-based compounds, porphyrin-based G-quadruplex inhibitors and perylenetetracarboxylic diimide, and wherein the detectable unit is selected from the group consisting of fluorescein or other fluorescein-related and/or -derived fluorophors such as 5-aminofluorescein or fluorescein-isothiocyanate (FITC), Oregon Green, Texas Red, Attodye, Cydye, Alexa647, Cy5, Cy3 or naphthofluorescein.

10. Pharmaceutical composition comprising a compound according to any of claims 1 to 9 and optionally at least one pharmaceutically acceptable excipient.

11. Use of a compound according to any of claims 1 to 9 or a composition of claim 10 in the manufacture of a medicament for diagnosing prostate cancer in a human or animal being.

12. Method of diagnosing in vivo prostate cancer in a human or animal subject comprising the steps of:
a) Administration of a compound comprising a targeting module and a detectable unit to said human or animal subject wherein the targeting module is capable of interacting with a prostate cancer-specific molecular marker;
b) Reaction of the targeting module of said compound with the prostate cancer-specific molecular marker;
c) Detection of the interaction between the prostate cancer-specific molecular marker and said compound by measuring outside the human or animal body a signal generated by the detectable unit; and
d) Deciding on the presence of prostate cancer on the basis of the signal measured in c).

13. Method of detecting in vivo a prostate cancer-specific molecular marker in a human or animal subject comprising the steps of:
a) Administration of a compound comprising a targeting module and a detectable unit to said human or animal subject wherein the targeting module is capable of interacting with a prostate cancer-specific molecular marker;
b) Reaction of the targeting module of said compound with the prostate cancer-specific molecular marker; and
c) Detection of the interaction between the prostate cancer-specific molecular marker and said compound by measuring a signal generated by the detectable unit.
